(19) United States
(12) Patent Application Publication
(10) Pub. No.: US 2015/0374307 A1
(43) Pub. Date: Dec. 31, 2015

(54) DATA ANALYSIS DEVICE, DATA ANALYSIS METHOD, AND DATA ANALYSIS PROGRAM

(71) Applicant: CASIO COMPUTER CO., LTD., Tokyo (JP)

(72) Inventor: Tomoaki Nagasaka, Tokyo (JP)

(21) Appl. No.: 14/753,309

(22) Filed: Jun. 29, 2015

(30) Foreign Application Priority Data
Jun. 30, 2014 (JP) 2014-133769

(51) Int. Cl. A61B 5/00; G01B 21/00; A61B 5/11; G01P 3/00

(52) U.S. Cl. CPC A61B 5/7278; G01P 3/00; G01B 21/00; A61B 5/1121; A61B 5/7264; A61B 5/486; A61B 5/7225

(57) ABSTRACT

A data analysis device which collects sensor data in time series from a sensor attached to a user during movement in a course having a plurality of sections where two adjacent sections are connected to each other and shapes along extended directions of the sections connected to each other are different from each other, estimates times of section change points based on the sensor data, estimates a movement speed at each section, based on times of each of the plurality of the section change points and a distance value of each section, calculates differences between movement speeds at two sections connected to each other, and adjusts the movement speeds to appropriate values by adjusting at least one of the plurality of section change points so as to reduce a value of sum total of the differences.

### CROSS-REFERENCE TO RELATED APPLICATION

This application is based upon and claims the benefit of priority from the prior Japanese Patent Application No. 2014-133769, filed Jun. 30, 2014the entire contents of which are incorporated herein by reference.

### BACKGROUND OF THE INVENTION

1. Field of the Invention

The present invention relates to a data analysis device, a data analysis method, and a data analysis program for visualizing and providing the motion status (exercise status) of a human body performing exercise.

2. Description of the Related Art

In recent years, because of rising health consciousness, more and more people are performing daily exercises, such as running, walking, and cycling, to maintain their wellness or improve their health conditions. In addition, an increasing number of people are aiming to participate in a race such as a marathon race through these daily exercises. These people are very conscious of and interested in measuring and recording various biological and exercise information by numerical values and data so as to grasp their own health conditions and exercise statuses. The people aiming to participate in a race have an objective of achieving a successful record in the race, and therefore are very conscious of and interested in efficient and effective training methods.

As indices for grasping an own health condition and exercise status based on numerical values and data measured during exercise, various indices are known. For example, in a case where the status and the form of running are quantitatively evaluated, information regarding the running speed, the stride, and the like can be used as an important and fundamental index. Here, as a method for measuring a running speed and a stride in a running exercise or a marathon race, for example, a method using positioning data and reception signals by GPS (Global Positioning System) is known. For example, Japanese Patent Application (Kohyo) Publication No. 10-325735 describes a technique where a stride per step is calculated based on a distance calculated based on the speed of a human body measured from the Doppler frequency of a carrier wave received by a GPS reception device worn on the human body, and the number of steps calculated based on vibration displacement detected by an acceleration sensor, and then the movement distance and the movement speed are calculated based on the stride which is updated by a GPS radio wave being periodically received, and the added number of steps.

Also, for example, Japanese Patent Application (Kohyo) Publication No. 2002-306660 describes a technique where the exercise status of a user is judged based on the current location of the user acquired by a GPS signal and, when the user is in an athletic field, the amount of the exercise, such as the movement distance, the movement speed, the consumed calorie, and the like of the user is calculated.

Each of the above-described patent applications discloses a method where the movement speed, the movement distance, and the stride of a human body are calculated and corrected based on positioning data and a reception signal acquired by GPS. However, in this method, GPS signals sometimes cannot be acquired completely or normally in a place where GPS radio waves are difficult to receive, such as indoor areas and places between buildings. In this case, the accuracy of the calculation of a movement speed, a movement distance, and a stride is significantly decreased, and therefore they sometimes cannot be fully utilized to accurately grasp, judge, and improve the exercise status.

### SUMMARY OF THE INVENTION

The present invention has an advantage of providing a data analysis device, a data analysis method, and a data analysis program by which the exercise status of a human body can be accurately estimated based on sensor data collected in time series from a sensor worn on the human body without use of GPS, so that the user can grasp, judge, and improve the exercise status.

In accordance with one aspect of the present invention, there is provided a data analysis device comprising: an section estimation portion which (a) collects sensor data in time series from a sensor attached to a user during movement in a course having a plurality of sections where two adjacent sections are connected to each other and shapes along extended directions of the sections connected to each other are different from each other, and (b) estimates times of a plurality of section change points each of which corresponds to time when the user has passed through a plurality of boundaries between each of the plurality of sections, based on changes of the sensor data corresponding to the shapes of the sections; a time-series velocity data generation portion which generates time-series velocity data indicating estimation values of movement speeds of the user at each section at elapsed time from movement start time of the user, based on times of each of the plurality of section change points and a distance value of each section; and a velocity data adjustment portion which (c) calculates differences between the movement speeds at two sections connected to each other among the plurality of sections, and (d) adjusts the movement speeds to appropriate values by adjusting at least one of the plurality of section change points so as to reduce a value of sum total of the differences.

In accordance with another aspect of the present invention, there is provided a data analysis method, comprising: a step of collecting sensor data in time series from a sensor attached to a user during movement in a course having a plurality of sections where two adjacent sections are connected to each other and shapes along extended directions of the sections connected to each other are different from each other; a step of estimating time of a plurality of section change points each of which corresponds to time when the user has passed through a plurality of boundaries between each of the plurality of sections, based on changes of the sensor data corresponding to the shapes of the sections; a step of generating time-series velocity data indicating estimation values of movement speeds of the user at each section at elapsed time from movement start time of the user, based on times of each of the plurality of estimated section change points and a distance value of each section; a step of calculating differences between the movement speeds at two sections connected to each other among the plurality of sections; and a step of adjusting the movement speeds to appropriate values by adjusting at least one of the plurality of section change points so as to reduce a value of sum total of the differences.

In accordance with another aspect of the present invention, there is provided a non-transitory computer-readable storage medium having a data analysis program stored thereon that is executable by a computer to actualize functions comprising: processing for collecting sensor data in time series from a sensor attached to a user during movement in a course having a plurality of sections where two adjacent sections are connected to each other and shapes along extended directions of the sections connected to each other are different from each other; processing for estimating time of a plurality of section change points each of which corresponds to time when the user has passed through a plurality of boundaries between each of the plurality of sections, based on changes of the sensor data corresponding to the shapes of the sections; processing for generating time-series velocity data indicating estimation values of movement speeds of the user at each section at elapsed time from movement start time of the user, based on times of each of the plurality of estimated section change points and a distance value of each section; processing for calculating differences between the movement speeds at two sections connected to each other among the plurality of sections; and processing for adjusting the movement speeds to appropriate values by adjusting at least one of the plurality of section change points so as to reduce a value of sum total of the differences.

The above and further objects and novel features of the present invention will more fully appear from the following detailed description when the same is read in conjunction with the accompanying drawings. It is to be expressly understood, however, that the drawings are for the purpose of illustration only and are not intended as a definition of the limits of the invention.

### BRIEF DESCRIPTION OF THE DRAWINGS

FIG. 1A and FIG. 1B are schematic structural diagrams showing an embodiment of an exercise support apparatus in which a data analysis device according to the present invention has been applied;

FIG. 2 is a flowchart showing an example of a control method (data analysis method) for the exercise support apparatus according to the embodiment;

FIG. 3A, FIG. 3B, and FIG. 3C are diagrams showing a user's running course and a relation between an integral value of angular velocity data generated while the user is running this course and time-series angular data in the data analysis method according to the embodiment;

FIG. 4 is a flowchart showing an example of cluster classification processing in the data analysis method according to the embodiment;

FIG. 5A, FIG. 5B, and FIG. 5C are conceptual diagrams for describing the cluster classification processing in the data analysis method according to the embodiment;

FIG. 6 is a flowchart showing an example of section estimation processing in the data analysis method according to the embodiment;

FIG. 7A, FIG. 7B, FIG. 7C, and FIG. 7D are first conceptual diagrams for describing the section estimation processing in the data analysis method according to the embodiment;

FIG. 8A, FIG. 8B, FIG. 8C, and FIG. 8D are second conceptual diagrams for describing the section estimation processing in the data analysis method according to the embodiment;

FIG. 9 is a flowchart showing an example of adjusting process in the data analysis method according to the embodiment;

FIG. 10A and FIG. 10B are first diagrams for describing the adjusting process in the data analysis method according to the embodiment;

FIG. 11 is a second diagram for describing the adjusting process in the data analysis method according to the embodiment;

FIG. 12 is a flowchart showing an example of change point position determination processing in the data analysis method according to the embodiment;

FIG. 13 is a diagram for describing the change point position determination processing in the data analysis method according to the embodiment; and

FIG. 14 is a diagram showing an effect of the adjusting process in the data analysis method according to the embodiment.

### DETAILED DESCRIPTION OF THE PREFERRED EMBODIMENTS

Hereinafter, embodiments of a data analysis device, a data analysis method, and a data analysis program according to the present invention will be described in detail with reference to the drawings.

Note that, in the following descriptions of the embodiments, a case is exemplified where a data analysis device according to the present invention is applied in an exercise support apparatus, and movement speeds and strides (stride length) when a user is running are estimated based on various types of data (sensor data) collected while the user is running around a running track on an athletic field or the like or running a predetermined running course or a marathon course, and then provided to the user.

(Exercise Support Apparatus)

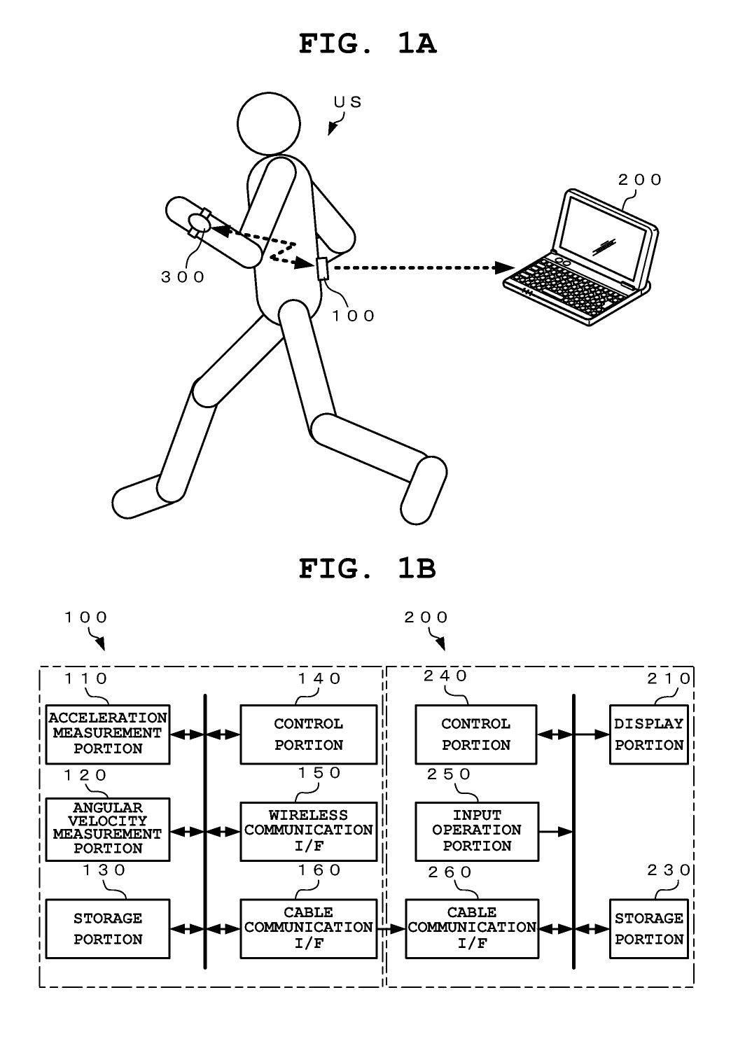

FIG. 1A and FIG. 1B are schematic structural diagrams showing an embodiment of an exercise support apparatus in which a data analysis device according to the present invention has been applied.

FIG. 1A is a conceptual diagram showing a state where a sensor device and the like applied in the exercise support apparatus of the embodiment have been worn on a human body, and FIG. 1B is a schematic block diagram showing the structures of the sensor device and the data analysis device.

The exercise support apparatus according to the embodiment of the present invention includes, for example, a sensor device 100 that is worn on the waist or the like of a user US, a control device 300 that is worn on a wrist or the like of the user US, and a data analysis device 200 that analyzes sensor data collected by the sensor device 100, as shown in FIG. 1A.

The sensor device 100 is a motion sensor having a function of measuring and accumulating various types of sensor data regarding the motion status of a human body performing an exercise involving movements such as a running exercise or a marathon.

In this embodiment, the sensor device 100 has been worn on the user's waist. However, the present invention is not limited thereto, and the sensor device 100 may be worn on any portion on a body axis passing through the center of a human body or a nearby portion. For example, the sensor device 100 may be worn on a chest, neck, abdomen, or the like excluding the waist portion.

Also, the method for wearing the sensor device 100 on the human body is not particularly limited, and various wearing methods can be adopted, such as a method where the sensor device 100 is clipped on exercise clothes, a method where the sensor device 100 is taped, and a method where the sensor device 100 is wound around a body by a belt.

Specifically, the sensor device 100 includes, for example, an acceleration measurement portion 110, an angular velocity measurement portion 120, a storage portion 130, a control portion 140, a wireless communication interface 150 (hereinafter referred to as “wireless communication I/F”), and a cable communication interface 160 (hereinafter referred to as “cable communication I/F”), as shown in FIG. 1B.

The acceleration measurement portion 110 measures the change ratio of the motion speed (acceleration) of the exercising user US. This acceleration measurement portion 110, which has a triaxial acceleration sensor, detects acceleration components (acceleration signals) in three axial directions orthogonal to each other, and outputs them as acceleration data. The angular velocity measurement portion 120 measures the change of the motion direction (angular velocity) of the exercising user US. This angular velocity measurement portion 120, which has a triaxial angular velocity sensor, detects angular velocity components (angular velocity signals) occurred in the rotational directions of rotational motions around the three axes that are orthogonal to each other and defining the above-described acceleration data, and outputs them as angular velocity data.

The sensor data (acceleration data, angular velocity data) acquired by the acceleration measurement portion 110 and the angular velocity measurement portion 120 is associated with time data generated in the control portion 140 described later, and stored in a predetermined storage area of the storage portion 130 described below.

The storage portion 130 stores sensor data (acceleration data, angular velocity data) acquired by the acceleration measurement portion 110 and the angular velocity measurement portion 120 in the predetermined storage area, in association with time data.

Note that the storage portion 130 may be partially or entirely in a form of a removable storage medium such as a memory card, and may be structured to be removable from the sensor device 100.

The control portion 140 is an arithmetic processing unit having a clocking function, such as a CPU (central processing unit) or a MPU (microprocessor), and executes a predetermined control program based on a predetermined operation clock. As a result, the control portion 140 controls various types of operations, such as a sensing operation by the acceleration measurement portion 110 or the angular velocity measurement portion 120, an operation of storing various types of data by the storage portion 130, a reading operation, and an operation of communicating with an external device and transferring data by the wireless communication I/F 150 or the cable communication I/F 160 described later.

The wireless communication I/F 150 receives at least an order signal transmitted from the control device 300 described later and instructing to start or end logging in the sensor device 100, and transmits it to the control portion 140. As a result, the start or the end of a sensing operation by the acceleration measurement portion 110 or the angular velocity measurement portion 120 is controlled, and sensor data acquired during the sensing operation are stored in time series in the predetermined storage area of the storage portion 130.

Note that, as a method for transmitting various types of signals between the sensor device 100 and the control device 300 in the wireless communication I/F 150, various types of wireless communication methods such as Bluetooth (registered trademark) and Wi-Fi (Wireless Fidelity (registered trademark)) can be adopted.

The cable communication I/F 160 has a function for transmitting at least sensor data stored in the storage portion 130 to the data analysis device 200 described later. As a result, in the data analysis device 200, predetermined data analysis processing in which the running speeds and the strides of the user US are estimated is performed. Note that, as a method for transmitting sensor data to the data analysis device 200 from the sensor device 100 in the cable communication I/F 160, various types of cable communication methods via a USB (Universal Serial Bus) standard communication cable (USB cable) and the like can be adopted.

The data analysis device 200 has a function for estimating movement speeds (running speeds) and strides based on various types of sensor data measured and accumulated by the sensor device 100 during the exercise of the user US, and providing them as indices (exercise indices) related to the exercise status of the human body.

Here, the data analysis device 200 may be a note-type or desktop-type personal computer or a portable information terminal such as a smartphone (sophisticated cellular phone) or a tablet terminal as long as it has a function for executing a data analysis program described later.

In a configuration where the data analysis program is executed using a cloud system on a network, the data analysis device 200 may be a communication terminal connected to the cloud system.

Specifically, the data analysis device 200 includes, for example, a display portion 210, a storage portion 230, a control portion (a time-series angular data generation portion, a cluster classification portion, a section estimation portion, a time-series velocity data generation portion, a velocity data adjustment portion, an exercise index providing portion) 240, an input operation portion 250, and a cable communication I/F 260, as shown in FIG. 1B.

The display portion 210 has a display panel of, for example, a liquid-crystal type capable of color display or a light-emitting-element type such as an organic EL element, and displays at least information related to an input operation using the input operation portion 250 described later or an analysis result based on sensor data, in a predetermined form.

Specifically, the display portion 210 displays, for example, sensor data (acceleration data, angular velocity data) stored in the storage portion 230 described later, graphs showing running speeds and strides calculated based on these sensor data, and various types of setting menus.

The storage portion 230 stores, in a predetermined storage area, sensor data transmitted from the sensor device 100 via the cable communication I/F 260 described later. Here, the sensor data are stored in the storage portion 230 in time series in association with a running method (contents of practice and the like) and a course condition (course type, running distance, corner angle, and the like)

These sensor data to be stored in the storage portion 230 may be those of one particular user, or be those of a plurality of users.

The storage portion 230 stores data that is used or generated when data or a graph showing running speeds and strides is generated or when various types of information is displayed on the display portion 210 by a predetermined control program or algorithm program being executed in the control portion 240.

Note that the storage portion 230 may store the control program and the algorithm program to be executed in the control portion 240.

Also, the storage portion 230 may be partially or entirely in a form of a removable storage medium such as a memory card, and may be structured to be removable from the data analysis device 200.

The control portion 240, which is an arithmetic processing unit such as a CPU and a MPU, executes a predetermined control program, and thereby controls various types of operations, such as the display of various types of information on the display portion 210, the transmission of sensor data from the sensor device 100 by the cable communication I/F 260 described later, and the storing and reading of sensor data in the storage portion 230.

By executing a predetermined algorithm program stored in the storage portion 130, the control portion 240 extracts sensor data for training and practice desired by the user US from the storage portion 230, and performs analysis processing of estimating running speeds and strides which are exercise indices.

Note that the control program and the algorithm program to be executed by the control portion 240 may be stored in advance in the control portion 240. Details of the data analysis method of the present embodiment will be described later.

The input operation portion 250 is an input means attached to the data analysis device 200, such as a keyboard, a mouse, a touchpad, and a touch panel. The user US uses this input operation portion 250 to select an arbitrary item or icon displayed on the display portion 210 or to indicate an arbitrary point on the screen, and thereby performs a function corresponding to the item or icon, or the point. For example, the input operation portion 250 is used to perform an input operation of selecting training or practice for which analysis processing is performed on sensor data stored in the storage portion 230. Note that the input means to be applied as the input operation portion 250 may be one of the various types of input means described above, or may be a plurality of input means.

The cable communication I/F 260 has a function for receiving at least sensor data transmitted from the above-described sensor device 100 and transmitting it to the storage portion 230. Here, as a technique for receiving sensor data from the sensor device 100 by the cable communication I/F 260, the above-described cable communication method using a USB cable may be adopted.

The control device 300 is connected at least to the sensor device 100 using a predetermined wireless communication method. By the user US operating the operation section of the control device 300, a command signal instructing the start or the end of logging is transmitted to the sensor device 100 from the control device 300. As a result, in the sensor device 100, the start or the end of a sensing operation by the acceleration measurement portion 110 or the angular velocity measurement portion 120 is controlled.

Here, as a technique for transmitting various types of signals between the control device 300 and the sensor device 100, the various types of wireless communication methods described above, such as Bluetooth (registered trademark) and Wi-Fi (Wireless Fidelity (registered trademark)), can be adopted.

Note that the control device 300 may have a function for displaying (notifying of) sensor data acquired by the sensor device 100, the operating status of the sensor device 100, time information, and the like, in addition to the above-described function for controlling a sensing operation by the above-described sensor device 100.

In the configuration of the exercise support apparatus according to the present embodiment, data communication between the sensor device 100 and the data analysis device 200 is made by cable communication and data communication between the sensor device 100 and the control device 300 is made by wireless communication. However, the present invention is not limited thereto.

That is, a configuration may be adopted in which data communication between the sensor device 100 and the data analysis device 200 is made by wireless communication or data communication between the sensor device 100 and the control device 300 is made by cable communication.

Also, a method may be adopted in which, by removable storage media such as memory cards constituting the storage portion 130 of the sensor device 100 and the storage portion 230 of the data analysis device 200 being switched, sensor data is transmitted to the data analysis device 200 from the sensor device 100.

In this embodiment, as the control device 300, the wristwatch-type (or wristband-type) device which is worn on a wrist of the user US has been described, as shown in FIG. 1A. However, the present invention is not limited thereto.

That is, the control devices may be a portable information terminal such as a smartphone or a dedicated terminal placed in, a pocket or worn on an upper arm portion. Also, a structure may be adopted in which an operation switch for instructing to start or end logging is provided on the body of the sensor device so that a device separated from the sensor device 100 is not required.

(Data Analysis Method)

Next, a control method (data analysis method) for the exercise support apparatus according to the present embodiment is described with reference to the drawings. Here, a series of control processing is described from processing for collecting and accumulating sensor data during exercise by the sensor device 100 to processing for estimating indices (running speeds and strides) related to an exercise status and providing them to the user by the data analysis device 200.

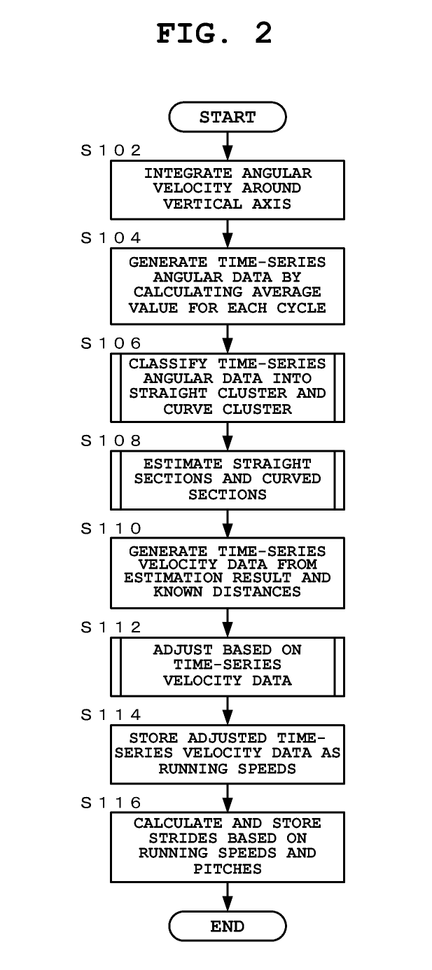

FIG. 2 is a flowchart showing an example of the control method (data analysis method) for the exercise support apparatus according to the present embodiment.

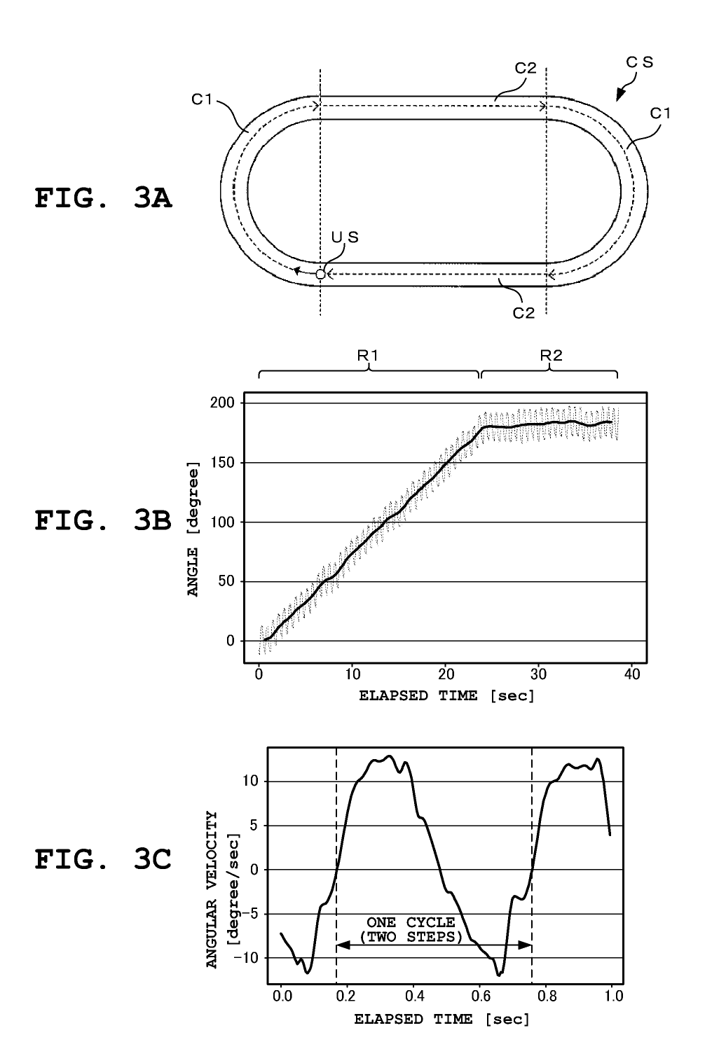

FIG. 3A, FIG. 3B, and FIG. 3C are diagrams showing the user's running course and a relation between an integral value of angular velocity data generated while the user is running this course and time-series angular data in the data analysis method according to the present embodiment.

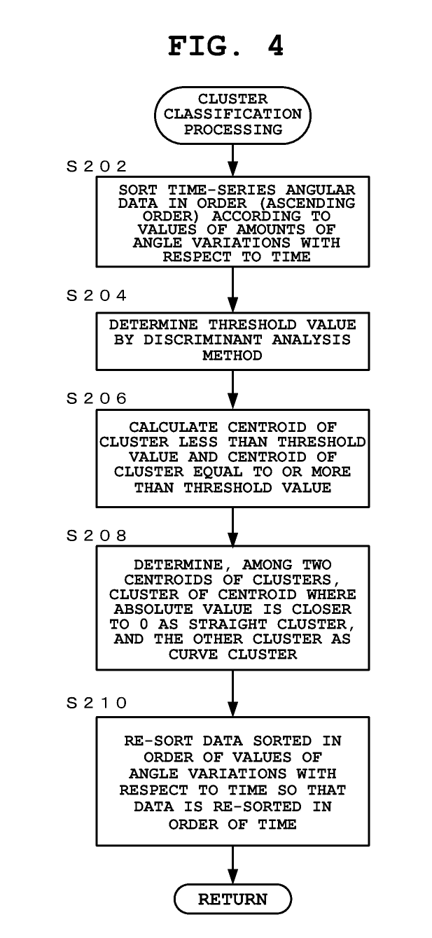

FIG. 4 is a flowchart showing an example of cluster classification processing in the data analysis method according to the present embodiment.

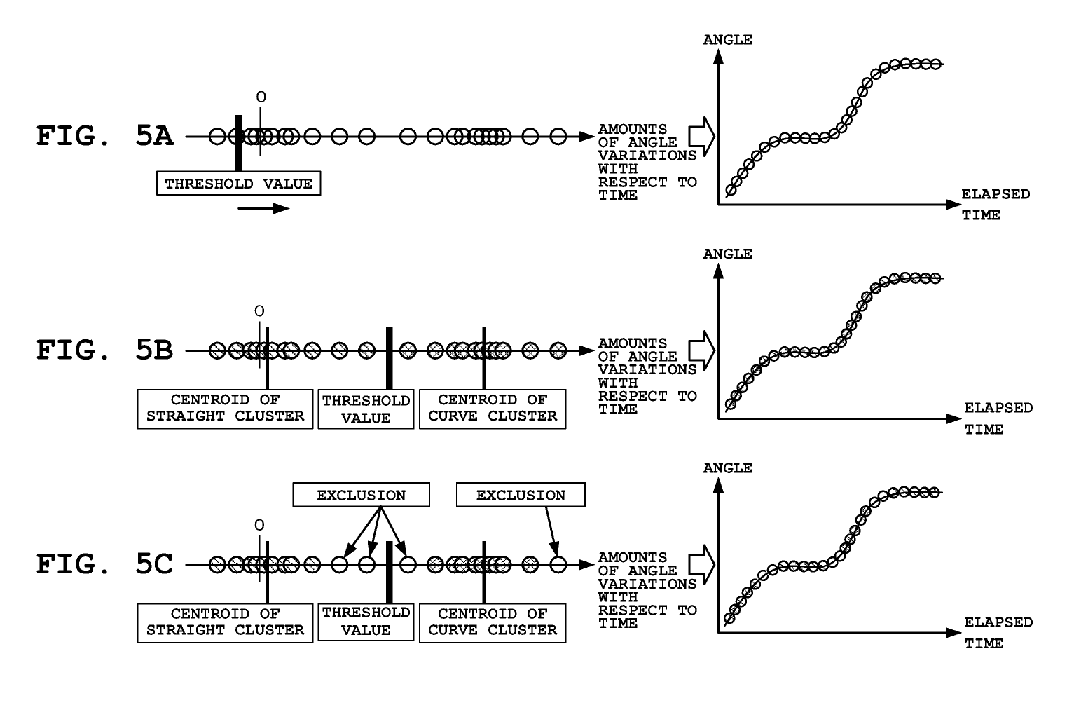

FIG. 5A, FIG. 5B, and FIG. 5C are conceptual diagrams for describing the cluster classification processing in the data analysis method according to the present embodiment.

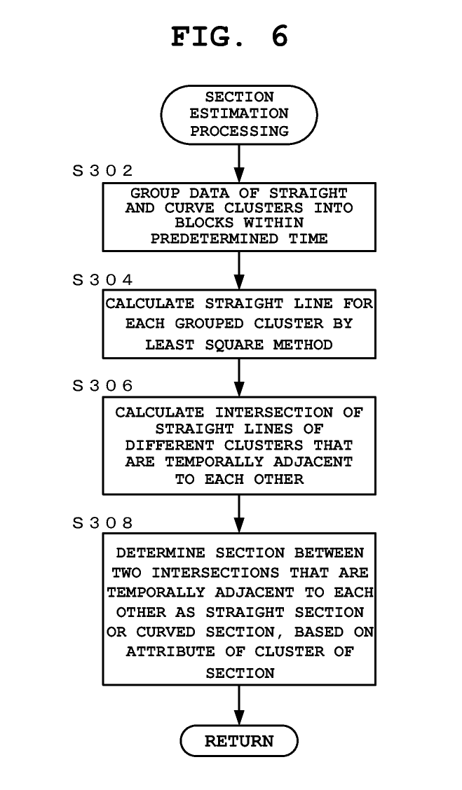

FIG. 6 is a flowchart showing an example of section estimation processing in the data analysis method according to the present embodiment.

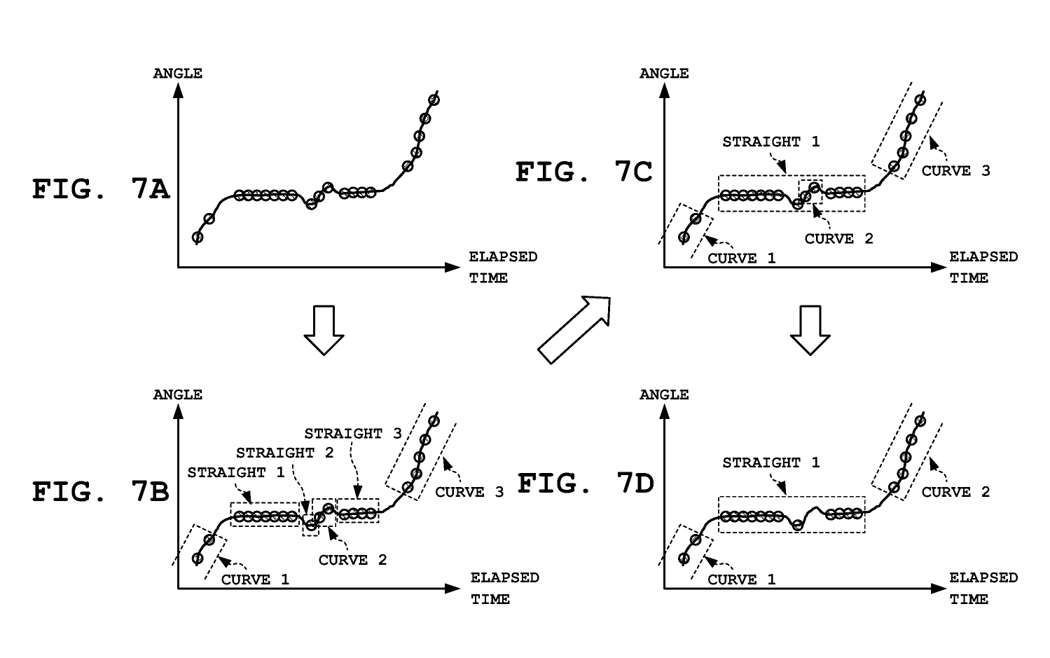

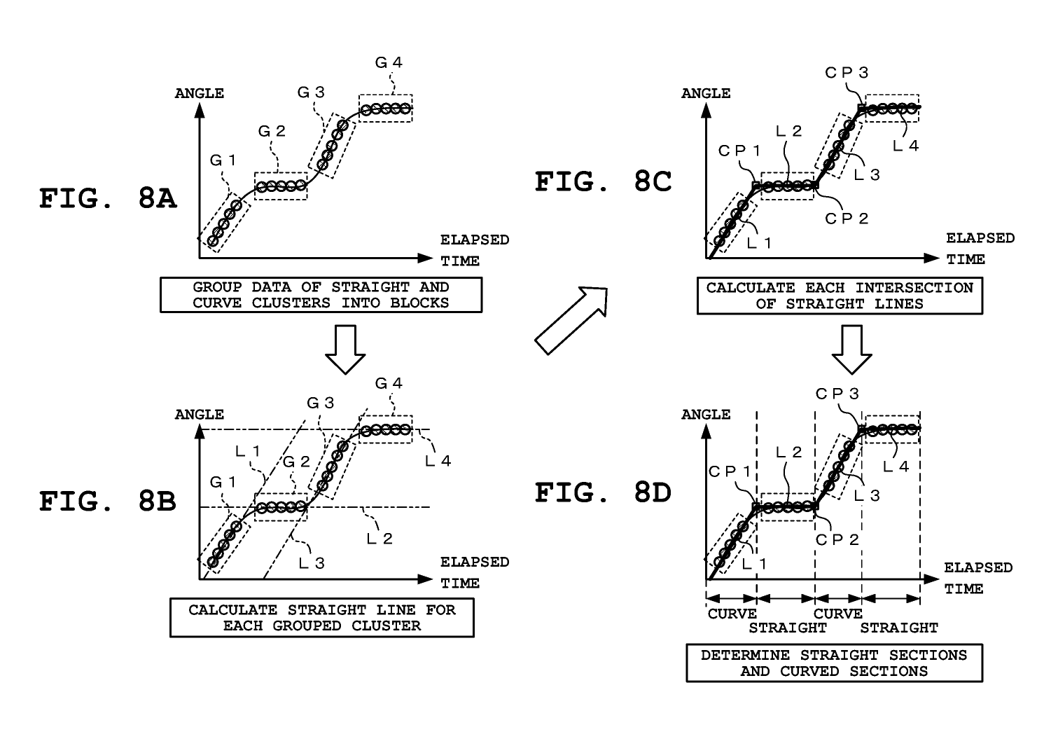

FIG. 7A, FIG. 7B, FIG. 7C, FIG. 7D, FIG. 8A, FIG. 8B, FIG. 8C, and FIG. 8D are conceptual diagrams for describing the section estimation processing in the data analysis method according to the present embodiment.

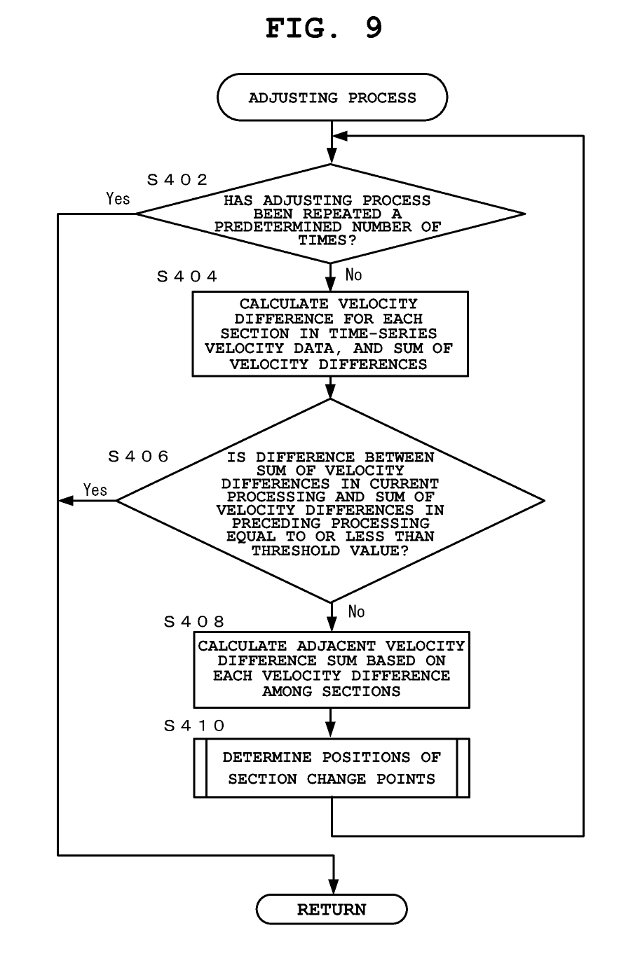

FIG. 9 is a flowchart showing an example of adjusting process in the data analysis method according to the present embodiment.

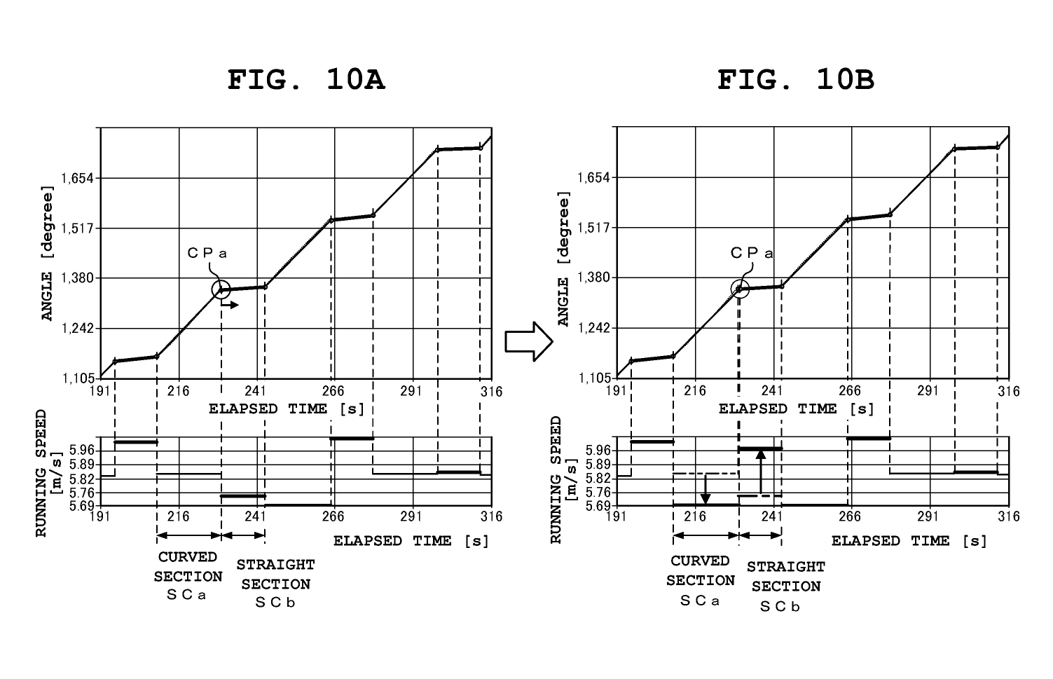

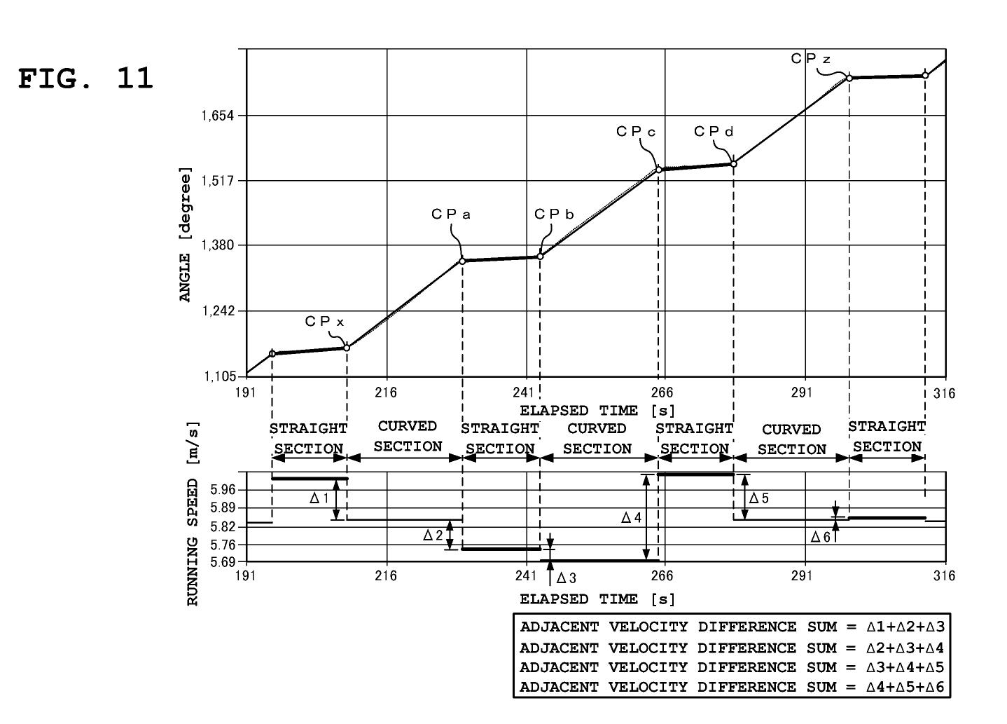

FIG. 10A, FIG. 10B, and FIG. 11 are diagrams for describing the adjusting process in the data analysis method according to the present embodiment.

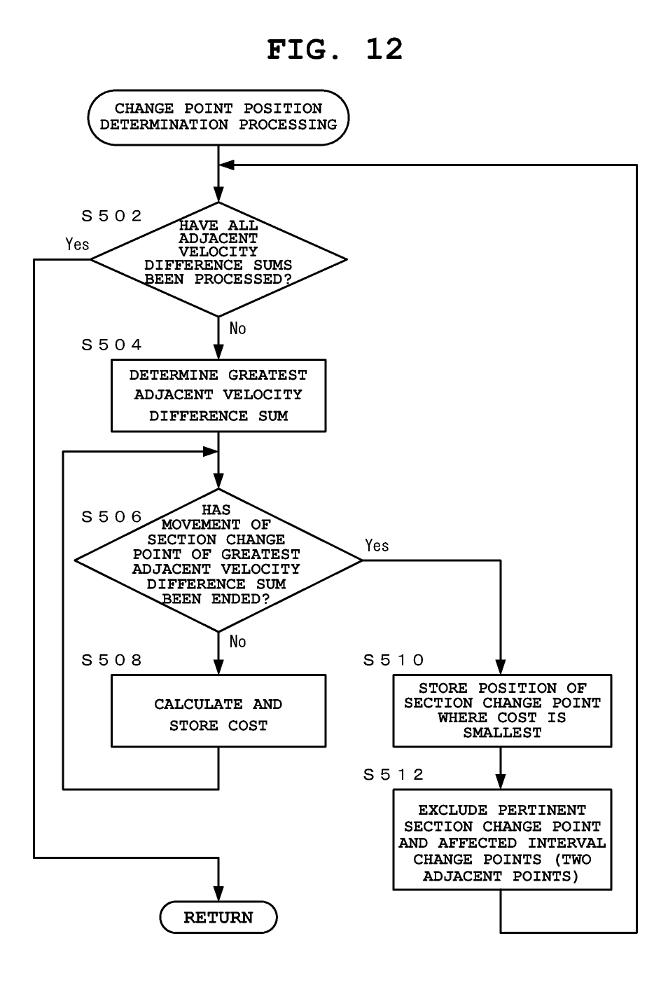

FIG. 12 is a flowchart showing an example of change point position determination processing in the data analysis method according to the present embodiment.

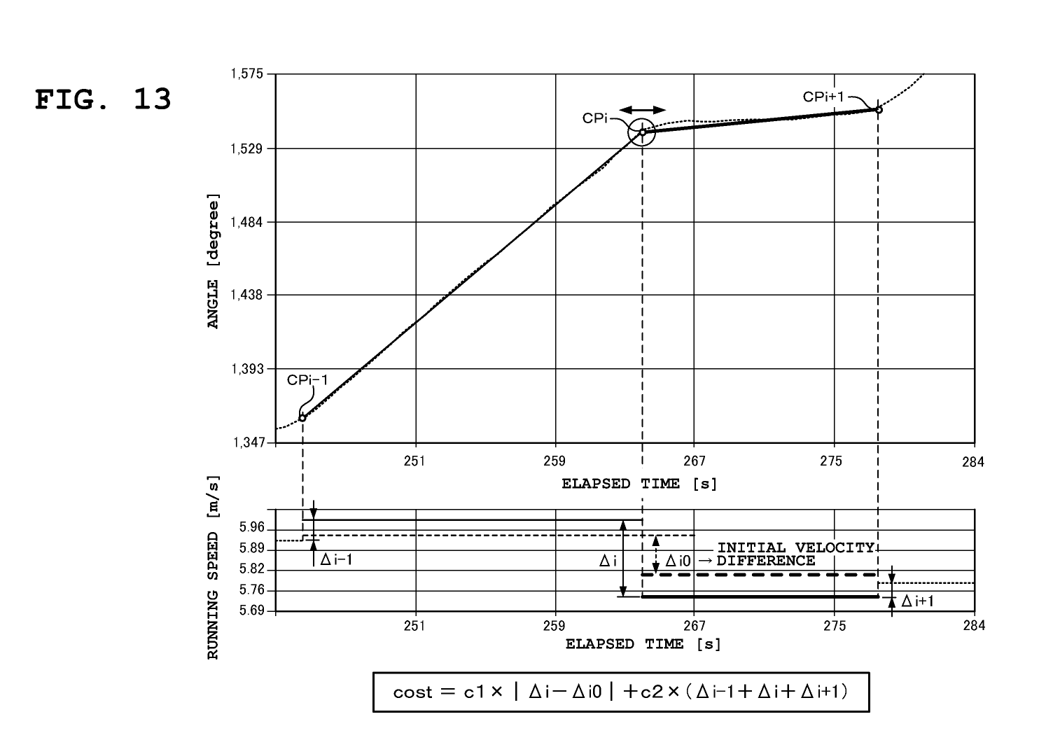

FIG. 13 is a diagram for describing the change point position determination processing in the data analysis method according to the present embodiment.

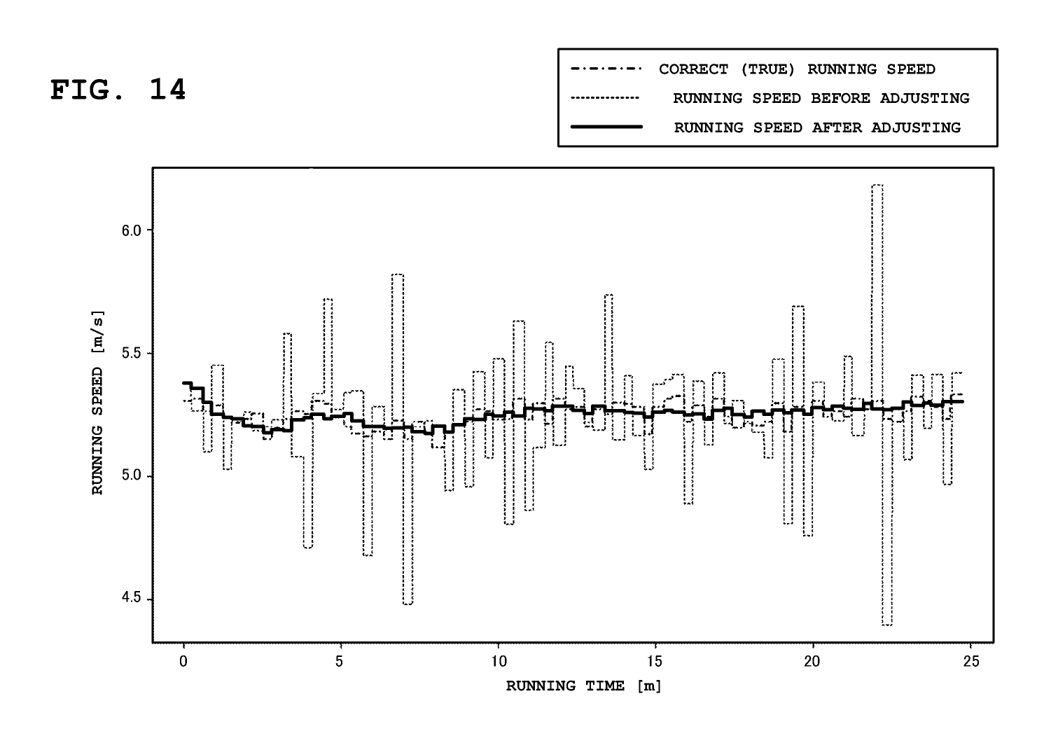

FIG. 14 is a diagram showing an effect of the adjusting process in the data analysis method according to the present embodiment.

The control method (data analysis method) for the exercise support apparatus according to the present embodiment mainly includes a step (sensor data collection step) of collecting and accumulating various types of sensor data related to an exercise status in a running exercise, and a step (index estimation step) of estimating indices (running speeds and strides) related to the exercise status based on the collected sensor data and providing them to the user US.

Here, the processing for estimating indices is actualized based on the predetermined algorithm program executed by the control portion 240 of the data analysis device 200.

In the sensor data collection step, first, the user US having the sensor device 100 worn on the waist runs a course where a plurality of sections (such as the straights and curves of a running track) whose shapes along the extending direction of the course are different from each other are continuous with each other and the entire distance and the distance of each section are known (confirmed), such as a running track on an athletic field, a predetermined running course or a marathon course, as shown in FIG. 1A.

Here, when starting the running exercise, the user US operates the control device 300 worn on a wrist or the like, whereby a command signal instructing to start logging is transmitted to the sensor device 100 from the control device 300.

Accordingly, the control portion 140 of the sensor device 100 starts the measurement of sensor data (acceleration data and angular velocity data) by the acceleration measurement portion 110 and the angular velocity measurement portion 120, and sequentially stores the data in the storage portion 130.

Then, when ending the running exercise, the user US operates the control device 300, whereby a command signal instructing to end the logging is transmitted to the sensor device 100 and the measurement of sensor data by the acceleration measurement portion 110 and the angular velocity measurement portion 120 is completed.

As a result, the sensor data indicating a motion status during the running exercise is acquired in association with time data.

Subsequently, the sensor device 100 and the data analysis device 200 are connected by, for example, a USB cable. As a result, the sensor data stored during the running exercise is transmitted to the data analysis device 200 from the sensor device 100, and stored in the storage portion 230.

Here, when transmitting the sensor data to the data analysis device 200 from the sensor device 100 (or while referring to the sensor data stored in the storage portion 230 by the use of the display portion 210), the user US inputs various information regarding the running exercise (exercise status) at the time of the acquisition of the sensor data into the data analysis device 200 by the use of the input operation portion 250.

Specifically, the running method (contents of practice and the like) and the course condition (the course type, the running distance, the corner angles, and the like) in the running exercise and item information including the user's name and the like are inputted into the data analysis device 200.

Next, the control portion 240 performs axial correction processing on the acceleration data among the sensor data stored in the storage portion 230.

Generally, the sensor device 100 worn on the body axis of a human body or a nearby portion is affected by the sway and tilt of the upper part of the body during an exercise such as a running exercise. Accordingly, a difference occurs between the axis of the gravity direction and the axis of acceleration in the vertical direction of the human body detected by the sensor device 100. Therefore, correction for offsetting a difference component between the axial directions, which is different for each clock time, is required to be made based on the value of angular velocity data acquired by the sensor device 100.

Specifically, in the axial correction processing, the control portion 240 first estimates a gravity direction for each clock time, based on the angular velocity data acquired by the sensor device 100. Subsequently, the control portion 240 rotates each axis of the acceleration data such that the estimated gravity directions and the vertical directions of the acceleration data coincide with each other, and thereby corrects the values of the acceleration data. The corrected acceleration data and the angular velocity data are stored in the predetermined storage area of the storage portion 230, as corrected sensor data.

Then, in the index estimation step, the control portion 240 analyzes the corrected sensor data, and calculates a pitch, a vertical motion, a foot landing time, and the like for each step in the running motions, as indices related to the exercise status in the running exercise. In addition, the control portion 240 performs processing for estimating the running speeds and the strides by calculating them.

In the present embodiment, a case where the user US runs a course CS shown in FIG. 3A is described for convenience of explanation. This course CS has curved sections C1 and straight sections C2 whose shapes along the extending direction of the course are different from each other and which are continuous with each other. At this course CS, the user US runs a couple of laps clockwise as shown by the arrows An example of this course is a running track on an athletic field.

However, courses where the present embodiment can be applied are not limited to such a running track. The same concept can be applied to a course having three or more sections (such as a straight section, a gently curved section, and a sharply curved section) whose shapes along the extending direction of the course are different from each other and which are continuous with each other, as long as its distance is known.

Specifically, in the index estimation processing, the user US first selects arbitrary corrected sensor data stored in the storage portion 230 by the use of the input operation portion 250 of the data analysis device 200.

As a result, the control portion 240 integrates angular velocity around a vertical axis in the corrected sensor data with respect to elapsed time, by executing a predetermined algorithm program (data analysis program), as shown in the flowchart of FIG. 2 (Step S102).

The vertical axis herein refers to an axis that is perpendicular to the ground and indicating the gravity direction. The control portion 240 extracts the data of angular velocity around this vertical axis from the corrected sensor data, and integrates it with respect to elapsed time. The result of the integration processing on the angular velocity data is represented by, for example, a broken line, as shown in FIG. 3B.

Then, the control portion 240 generates time-series angular data by calculating, with respect to the result of the integration processing on the angular velocity data, an average value of running motions for each cycle (Step S104).

This time-series angular data is represented by, for example, a solid line, as shown in FIG. 3B. The cycle herein refers to the period of one cycle of rotational motions around the body axis of user US from when the user US faces the front during the running exercise until when the user US faces the front again after taking two steps.

Specifically, in the angular velocity data which changes periodically, two steps in running motions indicate one cycle (a period during which the angle is changed once in each of the positive and negative directions), as shown in FIG. 3B. Alternatively, one cycle of running motions maybe calculated based on right and left foot landing timing detected from vertical acceleration data among the sensor data.

In the time-series angular data shown in FIG. 3B, an area R1 where the angle is increasing with the increase of the elapsed time corresponds to a period during which the user is running on the curved section C1 in FIG. 3A whose shape along the extending direction of the course is curved at a substantially constant curvature. Also, an area R2 where the angle is substantially constant (substantially same) regardless of the increase of the elapsed time corresponds to a period during which the user is running on the straight section C2 in FIG. 3A whose shape along the extending direction of the course is substantially straight.

The time-series angular data generated as described above is displayed on the screen of the display portion 210 in a predetermined form, such as a graphical form.

Then, the control portion 240 performs a series of cluster classification processing (clustering) for classifying the above-described time-series angular data into a straight cluster and a curve cluster (Step S106).

The cluster classification processing in the present embodiment is performed using a well-known method such as “discriminant analysis method (Otsu's binarization method)”.

Specifically, the control portion 240 first sorts (rearrange) the time-series angular data in ascending order according to the values of the amounts of angle variations with respect to a certain elapsed time, as shown in the flowchart of FIG. 4 (Step S202)

Subsequently, the control portion 240 determines a threshold value for favorably dividing the time-series angular data sorted in the order of the values of the amounts of angle variations with respect to a certain elapsed time into a plurality of clusters by usirig the discriminant analysis method, as shown in FIG. 5A (Step S204)

Here, in the discriminant analysis method (such as Otsu's binarization method), an angle to be the threshold value is sequentially changed (increased), by which a threshold value where the value of dispersion (substantially, dispersion among classes) is maximum in the distribution of the angular data is calculated, as shown in FIG. 5A and FIG. 5B.

Subsequently, with the determined threshold value as a criterion (boundary) the control portion 240 calculates the centroid of a cluster (cluster on the left side of the drawing) less than the threshold value in the distribution of the angular data and the centroid of a cluster (cluster on the right side of the drawing) equal to or more than the threshold value, as shown in FIG. 5B (Step S206).

Then, the control portion 240 determines, among the two calculated centroids, a cluster (cluster on the left side of the drawing) of a centroid where the absolute value of the angle is closer to 0 as a straight cluster, and determines the other cluster (cluster on the right side of the drawing) as a curve cluster, as shown in FIG. 5B (Step S208).

The classification of clusters and the determination of their attributions such as this are on the ground that, generally, angle variation indicating the sway and tilt of a human body when running on a straight section in a running exercise and the like is relatively small, and angle variation when running on a curved section is relatively large. Then, the control portion 240 re-sorts the angular data classified (determined) into the straight cluster and the curve cluster such that the state of being sorted in the order of the values of the amounts of angle variations with respect to a certain elapsed time is changed back to the state of being sorted in order of time (Step S210), ends the cluster classification processing, and returns to the flowchart shown in FIG. 2.

Note that a configuration may be adopted in which, after classifying the angular data into a straight cluster and a curve cluster at the above-described Step S208, the control portion 240 further performs processing of excluding, among the angular data included in each cluster, data that are far from the centroids, as shown in FIG. 5C.

Here, the data that are far from the centroids in the clusters are most likely data acquired at the boundary between the straight section and the curve section or data affected by a sudden movement. Accordingly, data that are, for example, two or more times farther from the centroids than the standard deviation of each cluster are excluded.

In the series of cluster classification processing (clustering) described above, the discriminant analysis method (Otsu's binarization method) has been adopted. However, the present invention is not limited to thereto. For example, a well-known method, such as “k-means clustering”may be adopted.

Then, the control portion 240 performs a series of section estimation processing for estimating straight sections and curved sections, based on the result of the above-described cluster classification processing (Step S108). Here, the section estimation processing to be performed in the present embodiment has an effect of removing and reducing the effect of noise and drift components in the sensor data collected by the sensor device 100, when it is applied to the time-series angle data.

In this embodiment, the time-series angle data has been classified into the two straight and curve clusters, based on the result of the above-described cluster classification processing at Step S106, as shown in, for example, FIG. 7A.

Note that the time-series angle data shown in FIG. 7A is an example of more realistic time-series angle data which is different from the ideal time-series angle data shown in FIG. 5A, FIG. 5B, and FIG. 5C and in which noise and drift components are included in its sensor data.

In the section estimation processing of the present embodiment, the control portion 240 groups the data of the straight and curve clusters classified in the time-series angle data into blocks within a predetermined elapsed time by the following procedure, as shown in the flowchart of FIG. 6 (Step S302).

Specifically, the control portion 240 first classifies temporally contiguous points in the same cluster in the time-series angular data into the same group, as shown in FIG. 7B.

In FIG. 7B, temporally contiguous points have been grouped into straights 1 to 3 and curves 1 to 3. Here, points included in straight 2 and curve 2 are abnormal values attributed to noise and drift components in the sensor data.

Subsequently, the control portion 240 compares groups in the same cluster, and combines groups that are not a predetermined amount of time Ta away from each other.

For example, when the groups of straights 1, 2, and 3 in the same cluster in FIG. 7B are not ten or more seconds away from each other, these groups are combined to one group (straight 1), as shown in FIG. 7C.

The predetermined amount of time Ta herein, which is used to judge whether or not to combine groups, should preferably be set to about 10 seconds in cases where the user runs around a running track as in the present embodiment.

Then, after combining the groups, the control portion 240 deletes a group within a predetermined amount of time Tb in each group, as shown in FIG. 7D.

In FIG. 7D, the group of curve 2 in the group of straight 1 has been deleted. The predetermined amount of time Tb herein, which is used to judge whether or not to delete a group, should preferably be set to about 10 seconds in cases where the user runs around a running track as in the present embodiment.

Then, the control portion 240 calculates a straight line for each grouped cluster by using the least square method (Step S304).

For example, for each cluster including groups G1 to G4 in time-series angular data in FIG. 8A, straight lines L1 to L4 indicating the change tendency of the angle of each cluster (groups G1 to G4) with respect to an elapsed time are calculated, as shown in FIG. 8B.

Then, the control portion 240 calculates the intersection of the straight lines of different clusters that are temporally adjacent to each other (Step S306).

For example, for the straight lines L1 to L4 calculated for each cluster (groups G1 to G4) in the time-series angular data in FIG. 8B, the intersection of the straight lines of different clusters that are temporally adjacent to each other is calculated, as shown in FIG. 8C.

In FIG. 8C, the intersection CP1 of the straight lines L1 and L2, the intersection CP2 of the straight lines L2 and L3, and the intersection CP3 of the straight lines L3 and L4 have been calculated.

Then, the control portion 240 determines each section between two intersections that are temporally adjacent to each other as a straight section or a curved section, based on the attribute of the cluster of this section, and stores them in the storage portion 230 (Step S308).

For example, sections between the adjacent intersections CP1 to CP3 calculated based on the straight lines Ll to L4 for each cluster (groups G1 to G4) in the time-series angular data in FIG. 8C are determined as straight sections or curved sections, based on the change tendencies of the angles of the groups G1 to G4 included in the clusters of the sections (or the tilt of the straight lines L1 to L4), as shown in FIG. 8D.

In FIG. 8D, the section between the intersections CP1 and CP2 has been determined as a straight section and the section between the intersections CP2 and CP3 has been determined as a curved section.

After the section estimation processing is ended, the control portion 240 returns to the flowchart of FIG. 2.

Note that, for convenience of explanation, the above-described intersection is referred to as “section change point” in the descriptions below.

As described above, the section change points (intersections CP1 to CP3) are to define each straight section and each curved section and to indicate the boundary between the straight section and the curved section.

Then, based on the time spent in the straight sections and the curved sections determined (estimated) in the above-described Step S108 and the known distance of each section, the control portion 240 calculates the running speeds during the running exercise by the following formula (1), and generates time-series velocity data (Step S110).

running speed (m/s)=section distance (m)/pent time (s) (1)

The time-series velocity data generated as described above is displayed on the screen of the display portion 210 in a predetermined form, such as a graphical form.

Subsequently, the control portion 240 performs a series of adjusting processes for adjusting the running speeds in the time-series velocity data generated at the above-described Step S110 (Step S112).

In this embodiment, the adjusting process is performed based on an assumption that, at least when a runner is running on continuous straight and curved sections such as a running track on an athletic field or the like, the running speed does not change rapidly, but changes slowly (smoothly).

Here, in the adjusting process in the present embodiment, a method is adopted in which a processing result gradually reaches a solution (true value) by the processing being repeated.

Specifically, in the adjusting process, the control portion 240 first judges whether the series of adjusting processes has been repeated a predetermined number of times, as shown in the flowchart of FIG. 9 (Step S402).

Then, when the processing has not been performed the predetermined number of times, the control portion 240 calculates each velocity difference among adjacent sections in the time-series velocity data, and the sum thereof (Step S404).

Conversely, when the series of adjusting processes has been repeated the predetermined number of times, the control portion 240 ends the repetition processing and returns to the flowchart of FIG. 2.

Subsequently, when the sum of velocity differences calculated at Step S404 in the preceding repetition processing exists, the control portion 240 judges whether the absolute value of a difference between the sum of the velocity differences in the current processing and that in the preceding processing is equal to or less than a predetermined threshold value (Step S406)

When the absolute value of the difference between the sums of the velocity differences is equal to or less than the predetermined threshold value, the control portion 240 ends the repetition processing and returns to the flowchart of FIG. 2.

That is, when the absolute value of the difference between the sums of the velocity differences is equal to or less than the predetermined threshold value, the processing result can be regarded to have reached a solution (true value) in the current and the preceding processing, and therefore the control portion 240 ends the adjusting process.

When the above-described conditions (Step S402 and Step S406) for ending the processing has not been satisfied, or in other words, when the adjusting process has not been performed the predetermined number of times and the absolute value of the difference between the sum of the velocity differences in the current processing and that in the preceding processing is more than the predetermined threshold value, the control portion 240 calculates, based on each velocity difference among the sections, the sum of differences among adjacent velocities (adjacent velocity difference sum) (Step S408).

Here, in time-series angle data (upper portion in the drawing) and the corresponding time-series velocity data (lower portion in the drawing) represented by a graph shown in FIG. 10A, when an section change point CPa in the time-series angle data is moved (which is represented by the arrow in the upper portion of the drawing), a velocity difference between a curved section SCa and a straight section SCb adjacent to each other and defined by the section change point CPa in the time-series velocity data is affected simultaneously (which is represented by the arrows in the lower portion of the drawing), as shown in FIG. 10B.

Therefore, not only the velocity difference of one portion (between particular sections), but also velocity differences among adjacent sections are required to be taken into consideration.

Accordingly, in the present embodiment, the sum of velocity differences among adjacent sections is calculated and used for the processing for adjusting running speeds.

For example, as shown in FIG. 11, the control portion 240 calculates each adjacent velocity difference sum A to D for each section change point CPa to CPd in time-series angular data (upper portion in the drawing) by using differences Δ1 to Δ6 among running speeds at sections in time-series velocity data (lower portion in the drawing)

In FIG. 11, in the case of the section change point CPa, the sum of a difference Δ2 between a running speed at a curved section and a running speed at a straight section at the section change point CPa, a difference Δ1 between a running speed at a curved section and a running speed at a straight section at the section change point CPx adjacent to the section change point CPa, and a difference Δ3 between a running speed at a curved section and a running speed at a straight section at the section change point CPb has been calculated as an adjacent velocity difference sum A for the section change point CPa.

Note that, although the details are omitted here, adjacent velocity difference sums B to D for the other section change points CPb to CPd are calculated in the same manner.

Then, after calculating an adjacent velocity difference sum for each section change point at the above-described Step S408, the control portion 240 performs a series of change point position determination processing for determining proper positions of the section change points in the descending order of the adjacent velocity difference sums (Step S410)

Specifically, in the change point position determination processing, the control portion 240 first judges whether the series of change point position determination processing has been performed on all of the adjacent velocity difference sums, as shown in the flowchart of FIG. 12 (Step S502). When the processing has been performed on all of the adjacent velocity difference sums, the control portion 240 ends the change point position determination processing, and returns to the flowchart of FIG. 9.

Conversely, when the processing on all of the adjacent velocity difference sums has not been completed, the control portion 240 determines (extracts) the greatest adjacent velocity difference sum among the adjacent velocity difference sums (Step S504).

Subsequently, the control portion 240 calculates a specific parameter when the section change point of the determined greatest adjacent velocity difference sum is moved in a predetermined range.

Specifically, the control portion 240 calculates each “cost” that is a parameter represented by the following equation (2) while moving the section change point CPi of the greatest adjacent velocity difference sum in a predetermined range (which is indicated by arrows in the upper portion of the drawing), and sequentially stores the “costs” in the predetermined storage area of the storage portion 230 in association with the current position of the section change point CPi (moved position), as shown in FIG. 13 (Step S508).

Note that the predetermined range within which the section change point is moved should preferably be set to, when the sum of the lengths of straight and curved sections adjacent to the target section change point is 1, about ±0.1.

cost=c1×|ΔI−Δi0|+c2×(Δi−1+ΔI+Δi+1) (2)

Here, Δi−1, Δi, and Δi+1 are each an absolute value of a velocity difference between a movement speed at a temporally preceding section and a movement speed at a temporally subsequent section, at section change points CPi−1, CPi, and CPi+1. Δi0 is the initial value of the velocity difference, and c1 and c2 are coefficients.

In equation (2) representing “cost”the first term is the absolute value of a difference between the velocity difference Δi when the section change point CPi of the greatest adjacent velocity difference sum is moved and the velocity difference Δi0 that is the initial value.

Accordingly, the decrease of “cost” affects the section change point CPi such that it is not changed from the position of the initial value.

Also, in equation (2), the second term is the adjacent velocity difference sum (the sum of the velocity difference Δi at the section change point CPi and the velocity differences Δi−1 and Δi+1 at the section change points CPi−1 and CPi+1 on the sides thereof) at the section change point CPi.

Accordingly, the decrease of “cost” also affects such that the velocity differences among the adjacent sections are eliminated (or decreased) and a smooth change is made.

In equation (2), processing for adjusting the effects of these two terms (2) is performed by the values of the coefficients c1 and c2 being adjusted.

Specifically, in a case where the contents of practice when target sensor data to be analyzed in the present embodiment is acquired are running motions such as pace running where the velocity is basically not much changed, the values of the coefficients c1 and c2 are set such that the value of c2 is relatively high, such as c1=1 and c2=2, so that the velocity differences among the adjacent sections are eliminated (decreased) and a smooth change is made.

On the other hand, in a case where the contents of practice are buildup running, section training, etc., the values of the coefficients c1 and c2 are set such that the value of c1 is relatively high, such as c1=2 and c2=1, so that the effect of the initial value is increased, based on an assumption that the velocity difference is changed rapidly.

In addition, in order to address the rapid velocity change, an upper limit may be provided to the velocity difference in accordance with the contents of the practice.

The control portion 240 repeatedly performs the above-described processing for calculating and storing “cost” until the movement of the section change point CPi of the greatest adjacent velocity difference sum within the predetermined range is ended (Step S506)

Then, when the movement of the section change point CPi of the greatest adjacent velocity difference sum within the predetermined range is ended, the control portion 240 determines, as the position of the section change point CPi, a movement position where “cost” at each movement position of the section change point CPi which has been stored in the storage portion 230 by equation (2) is smallest, and stores it in the predetermined storage area of the storage portion 230 (Step S510).

Subsequently, the control portion 240 excludes the section change point where “cost” is smallest, which has been described at Step S510, and first and second adjacent section change points positioned on the sides thereof (a total of four points) (Step S512), and then returns to Step S502 to repeat the series of change point position determination processing described above.

That is, the control portion 240 performs the series of change point position determination processing in the descending order of the adjacent velocity difference sums of the section change points. However, as described above, when a target section change point is moved, the velocities of the adjacent sections are affected thereby, as shown in FIG. 10A and FIG. 10B. Therefore, in order to completely eliminate this effect, adjacent (first adjacent and second adjacent) section change points are repeatedly excluded from the processing.

Then, when the series of change point position determination processing is performed on all of the adjacent velocity difference sums, the control portion 240 returns to the flowchart of FIG. 9. In addition, when the series of adjusting processes is repeated the predetermined number of times, the control portion 240 ends the adjusting process and returns to the flowchart of FIG. 2.

Next, the control portion 240 stores, as running speeds, the value of each straight section and the value of each curved section in the time-series velocity data defined based on the positions of the section change points determined by the above-described adjusting process (Step S112), in the predetermined storage area of the storage portion 230 (Step S114).

Here, as shown in FIG. 14, according to the verification of the effect of the adjusting process in the present embodiment, running speeds (represented by a solid line in the drawing) after the adjusting process are much closer to the correct running speeds (true values: represented by a dashed dotted line in the drawing) as compared to running speeds (represented by a dotted line in the drawing) before the adjusting process.

Then, the control portion 240 calculates strides for each of the straight sections and the curved sections during the running exercise by the following equation (3), based on the running speeds acquired as described above and pitches calculated continuously, and stores them in the predetermined storage area of the storage portion 230 (Step S116).

stride (m/step)=running speed (m/s)/pitch (step/s) (3)

As described above, in the present embodiment, running speeds and strides when the user is running a course where the distance is known (confirmed) and a plurality of sections having different angles are included, such as a running track on an athletic field having straights and curves, can be accurately estimated based only on sensor data collected in time series by the motion sensor, without using GPS.

Here, in this embodiment, time-series angle data generated based on angular velocities around a vertical axis among collected sensor data is clustered, and section change points defining straight sections and curved sections are estimated based on the result thereof.

Then, time-series velocity data generated based on the estimation result is adjusted such that velocity changes in each section are smoothed, and running speeds and strides in each section are accurately estimated based on the adjusted time-series velocity data.

Therefore, according to the present embodiment, indices (running speeds and strides) related to the exercise status of the running user US can be automatically and accurately estimated, and the result thereof can be displayed on the screen of the display portion 210 in the form of a graph or numerical values, whereby the user US can accurately grasp the exercise status and use it for judgment and improvement.

In the above-described embodiment, for convenience of explanation, the case has been described in which the user US runs a couple of laps around a running track on an athletic field. However, the present invention is not limited thereto.

Even when the user runs an arbitrary running course, a marathon course, etc., the straight sections and the curved sections can be accurately estimated as long as it is a course where the distance is known (confirmed) and a plurality of sections having different angles are included, whereby indices (running speeds and strides) related to an exercise status during the running exercise can be provided to the user.

While the present invention has been described with reference to the preferred embodiments, it is intended that the invention be not limited by any of the details of the description therein but includes all the embodiments which fall within the scope of the appended claims.

What is claimed is:

1. A data analysis device comprising:
an section estimation portion which
(a) collects sensor data in time series from a sensor attached to a user during movement in a course having a plurality of sections where two adjacent sections are connected to each other and shapes along extended directions of the sections connected to each other are different from each other, and
(b) estimates times of a plurality of section change points each of which corresponds to time when the user has passed through a plurality of boundaries between each of the plurality of sections, based on changes of the sensor data corresponding to the shapes of the sections;
a time-series velocity data generation portion which generates time-series velocity data indicating estimation values of movement speeds of the user at each section at elapsed time from movement start time of the user, based on times of each of the plurality of section change points and a distance value of each section; and
a velocity data adjustment portion which
(c) calculates differences between the movement speeds at two sections connected to each other among the plurality of sections, and
(d) adjusts the movement speeds to appropriate values by adjusting at least one of the plurality of section change points so as to reduce a value of sum total of the differences.

2. The data analysis device according to claim 1, further comprising:
an exercise index providing portion which provides, as an exercise index, an index based on the appropriate values of the movement speeds at each section.

3. The data analysis device according to claim 1, wherein the velocity data adjustment portion adjust time of each section change point based on
(i) a change in the difference between the movement speeds at the two sections temporally adjacent to each other when the time of at least one of the plurality of section change points is adjusted,
(ii) a change in a difference between moving speeds at one section temporally preceding an other section among the two sections and an section temporally adjacent to and temporally preceding the one section, or
(iii) a change in a difference between moving speeds at the other section temporally subsequent to the one section among the two sections and an section temporally adjacent to and temporally subsequent to the other section.

4. The data analysis device according to claim 3, wherein the velocity data adjustment portion adjusts time of a first section change point CPi in order to achieve a minimum cost value obtained by equation (1)
cost=c1×|ΔI−Δi0|+c2×(Δi−1+ΔI+Δi+1) (1)
where the first section change point estimated by the section estimation portion is CPi,
a second section change point of time earlier than the first section change point CPi and adjacent to the first section change point CPi is CPi−1,
a third section change point of time later than the first section change point CPi and adjacent to the first section change point CPi is CPi+1,
an absolute value of a difference between estimation values of movement speeds at sections temporally preceding and subsequent to the first section change point CPi before adjusting time of section change points is Δi0,
an absolute value of a difference between estimation values of movement speeds at sections temporally preceding and subsequent to the second section change point CPi−1 after adjusting time of the section change points is Δi−1
an absolute value of a difference between estimation values of movement speeds at the sections temporally preceding and subsequent to the first section change point CPi after adjusting time of the section change points is ΔI,
an absolute value of a difference between estimation values of movement speeds at sections temporally preceding and subsequent to the third section change point CPi+1 after adjusting time of the section change points is Δi+1and
c1 and c2 are constants.

5. The data analysis device according to claim 1, further comprising:
a time-series angular data generation portion which generates time-series angular data indicating a plurality of values of angles with respect to predetermined direction among traveling directions of the user on the course for each elapsed time, based on the sensor data,
wherein the section estimation portion estimates the time of the plurality of section change points based on differences among values of change amounts of the angles with respect to a certain elapsed time in the time-series angular data.

6. The data analysis device according to claim 5, further comprising:
a cluster classification portion which classifies the plurality of values of the angles in the time-series angular data into a plurality of clusters where distributions of the values of the change amounts of the plurality of values of the angles with respect to the elapsed time are different from each other,
wherein the section estimation portion estimates the section change points based on classification to the plurality of clusters by the cluster classification section.

7. The data analysis device according to claim 6, wherein the cluster classification portion
classifies the plurality of values of the angles into the plurality of clusters based on a result of rearrangement of the plurality of values of the angles in the time-series angular data in order of the values of the change amounts for each certain elapsed time, and
determines an attribute corresponding to a shape of each of the plurality of clusters along the extended direction of the course, based on center values in the distributions of the values of the change amounts of the plurality of values of the angles with respect to the elapsed time in the plurality of clusters.

8. The data analysis device according to claim 6, wherein the section estimation portion
calculates an intersection of straight lines indicating a change tendency of the time-series angular data with respect to the elapsed time in each of two temporally adjacent clusters among the plurality of clusters, and
estimates a plurality of intersections for the plurality of clusters as the plurality of section change points.

9. The data analysis device according to claim 1, wherein the sensor includes at least an angular velocity sensor which outputs angular velocity data as the sensor data, and is worn on a body axis of body of the user or a nearby portion,
wherein the time-series angular data generation portion generates the time-series angular data by integrating the angular velocity data with respect to the elapsed time and calculating, for a result of integration of the angular velocity data, an average value of rotational motions around the body axis of the user for each cycle.

10. A data analysis method, comprising:
a step of collecting sensor data in time series from a sensor attached to a user during movement in a course having a plurality of sections where two adjacent sections are connected to each other and shapes along extended directions of the sections connected to each other are different from each other;
a step of estimating time of a plurality of section change points each of which corresponds to time when the user has passed through a plurality of boundaries between each of the plurality of sections, based on changes of the sensor data corresponding to the shapes of the sections;
a step of generating time-series velocity data indicating estimation values of movement speeds of the user at each section at elapsed time from movement start time of the user, based on times of each of the plurality of estimated section change points and a distance value of each section;
a step of calculating differences between the movement speeds at two sections connected to each other among the plurality of sections; and
a step of adjusting the movement speeds to appropriate values by adjusting at least one of the plurality of section change points so as to reduce a value of sum total of the differences.

11. The data analysis method according to claim 10, further comprising:
a step of providing, as an exercise index, an index based on the appropriate values of the movement speeds at each section.

12. The data analysis method according to claim 11, wherein the step of adjusting the movement speeds includes a step of adjusting time of each section change point based on
(i) a change in the difference between the movement speeds at the two sections temporally adjacent to each other when the time of at least one of the plurality of section change points is adjusted,
(ii) a change in a difference between moving speeds at one section temporally preceding an other section among the two sections and an section temporally adjacent to and temporally preceding the one section, or
(iii) a change in a difference between moving speeds at the other section temporally subsequent to the one section among the two sections and an section temporally adjacent to and temporally subsequent to the other section.

13. The data analysis method according to claim 10, further comprising:
a step of generating time-series angular data indicating a plurality of values of angles with respect to predetermined direction among traveling directions of the user on the course for each elapsed time, based on the sensor data,
wherein the step of estimating the time of the plurality of section change points includes a step of estimating the time of the plurality of section change points based on differences among values of change amounts of the angles with respect to a certain elapsed time in the time-series angular data.

14. The data analysis method according to claim 13, further comprising:
a step of classifying the plurality of values of the angles in the time-series angular data into a plurality of clusters where distributions of the values of the change amounts of the plurality of values of the angles with respect to the elapsed time are different from each other,
wherein the step of estimating the time of the plurality of section change points includes a step of estimating the time of the plurality of section change points based on classification to the plurality of clusters.

15. The data analysis method according to claim 14, wherein the step of classifying into the plurality of clusters includes a step of classifying the plurality of values of the angles into the plurality of clusters based on a result of rearrangement of the plurality of values of the angles in the time-series angular data in order of the values of the change amounts for each certain elapsed time, and determining an attribute corresponding to a shape of each of the plurality of clusters along the extended direction of the course, based on center values in the distributions of the values of the change amounts of the plurality of values of the angles with respect to the elapsed time in the plurality of clusters.

16. The data analysis method according to claim 14, wherein the step of estimating the time of the plurality of section change points includes a step of calculating an intersection of straight lines indicating a change tendency of the time-series angular data with respect to the elapsed time in each of two temporally adjacent clusters among the plurality of clusters, and estimating a plurality of intersections for the plurality of clusters as the plurality of section change points.

17. A non-transitory computer-readable storage medium having a data analysis program stored thereon that is executable by a computer to actualize functions comprising:
processing for collecting sensor data in time series from a sensor attached to a user during movement in a course having a plurality of sections where two adjacent sections are connected to each other and shapes along extended directions of the sections connected to each other are different from each other;
processing for estimating time of a plurality of section change points each of which corresponds to time when the user has passed through a plurality of boundaries between each of the plurality of sections, based on changes of the sensor data corresponding to the shapes of the sections;
processing for generating time-series velocity data indicating estimation values of movement speeds of the user at each section at elapsed time from movement start time of the user, based on times of each of the plurality of estimated section change points and a distance value of each section;
processing for calculating differences between the movement speeds at two sections connected to each other among the plurality of sections; and
processing for adjusting the movement speeds to appropriate values by adjusting at least one of the plurality of section change points so as to reduce a value of sum total of the differences.

18. The non-transitory computer-readable storage medium according to claim 17, further comprising:
processing for providing, as an exercise index, an index based on the appropriate values of the movement speeds at each section.

19. The non-transitory computer-readable storage medium according to claim 17, further comprising:
processing for generating time-series angular data indicating a plurality of values of angles with respect to predetermined direction among traveling directions of the user on the course for each elapsed time, based on the sensor data,
wherein the processing for estimating the time of the plurality of section change points includes processing for estimating the time of the plurality of section change points based on differences among values of change amounts of the angles with respect to a certain elapsed time in the time-series angular data.

20. The non-transitory computer-readable storage medium according to claim 19, further comprising:
processing for classifying the plurality of values of the angles in the time-series angular data into a plurality of clusters where distributions of the values of the change amounts of the plurality of values of the angles with respect to the elapsed time are different from each other,
wherein the processing for estimating the time of the plurality of section change points includes processing for estimating the time of the plurality of section change points based on classification to the plurality of clusters.
